(19) United States
(12) Patent Application Publication
(10) Pub. No.: US 2005/0288668 A1
(43) Pub. Date: Dec. 29, 2005

(54) Spinal column support system

(76) Inventor: Bernhard Brinkhaus, Oetwil an der Limmat (CH)

Correspondence Address: DENNISON, SCHULTZ, DOUGHERTY & MACDONALD, 1727 KING STREET, SUITE 105, ALEXANDRIA, VA, 22314, US

(21) Appl. No.: 10/517,606

(22) PCT Filed: Jun. 24, 2002
(86) PCT No.: PCT/EP02/06972

§ 371 Date: Dec. 21, 2004

(51) Int. Cl. A61B 17/70

(52) U.S. Cl. 606/61

(57) ABSTRACT

A spinal column support system, including a bone screw with an axially displaceable embodiment arranged above the bone screw shaft, a plate or rod arrangement with at least one opening into which the bone screw is screwed and an upper and lower disc which are mounted in the plate or rod at a distance from each other one over the other in a displaceable and positionable manner, and each including a hole through which the bone screw (16) is passed.

### TECHNICAL FIELD

The present invention relates to the field of spinal column support systems. These serve to cure or alleviate spinal column problems or spinal column disorders. More precisely, the invention relates to a system, wherein single vertebrae are either securely fixed via a plate/screw system or have a limited degree of residual movement with respect to each other within a defined movement space.

### PRIOR ART

In the systems currently found on the market, the vertebrae are fixedly connected to each other via a support system. These systems always require a straight line for all of the screws screwed into the individual vertebrae, so that the connection rods or connection plates can be integrated therein. In practice, this is not possible owing to the arrangement and variety of human vertebrae and the degree of accuracy that the surgeon can achieve. The screws can be forced into alignment by means of only one operation in spinal biomechanics, which in fact is not generally desired, in that different vertebrae are rotated by means of a protruding screw. Permanent, undesired clamps are thus implanted in the spinal system.

Moreover, the geometry of the used circular rods provides a poor static supporting function. Circular rods are not used as supports in industry.

An example of a known support system, wherein the vertebrae are securely fixed, is described in DE 195 10 543 C2. A screw comprises a bone screw shaft at one end and a threaded section at the other end, a nut-like base plate being screwed onto the threaded section. The base plate comprises a groove on the surface opposite the bone screw shaft, an attachment bar can be inserted into the groove for the purposes of connecting to another such device.

In current rod and slot systems, a screw and the vertebra fixedly connected thereto are rotated such that the screw is accommodated in the narrow slot or rod line. By reason of this, there are no simple starting situations for the surgeon because he does not know which forces travel into the spinal column system and how they are acting. As a result, the patient can suffer permanent pain for a long time.

A spinal column support system is known from WO 95/27444 A1 and comprises an elongated longitudinal plate arrangement having longitudinal slots in each of which a bone screw is disposed in a displaceable manner. Hemispherical nuts provided with eccentric openings are located on the bone screw above and below the plate arrangement and are tightened with the plate arrangement by a counter-nut. Only the tilting movement of the plate arrangement is adjustable by rotating the hemispherical nuts. The hemispherical nuts cannot be attached easily which means the system cannot have a wide variety of applications.

A series of very important requirements are placed upon a suitable and good biomechanical vertebrae support system, i.e., adapted particularly to human anatomy, and upon the possibilities of adjusting and aligning the vertebrae screws in the operating room:
The bone screws used must have a good resistance to being pulled out.
They must be able to be removed at a later date if need be.
The exiting part of the screw must be constructed in such a manner that certain inclined positions of the bone screw can be corrected.
Therefore, the bone screw must be multi-axial.
At the transition of the bone screw to a perpendicular support plate, the screw must have a degree of movement so that the vertebra can effect small conical movements in the intervertebral disk region.
Therefore, the ideal bone screw must be multi-axial and have a small degree of conical movement.
The perpendicular support system must be a plate system in which a very narrow gap is not provided for receiving screws but rather a zone is provided in which the bone screw can be fixedly screwed in a perpendicular manner.
Minute adjustments of the vertebrae spacing before the final tightening is likewise a great help for the surgeon and patient.

The currently known systems do not fulfill all of these requirements, wherein the complicated anatomical conditions and the exact placement of the bone vertebrae screws, which cannot be achieved, are the deciding factors.

### ILLUSTRATION OF THE INVENTION

It is thus the object of the invention to create a vertebrae support system that overcomes these difficulties and obviates the negative influencing factors on the spinal column system and has a positive influence on the healing process in addition to the purely supporting function.

This object is achieved for a spinal column support system by the features of Claim 1. Advantageous developments of the spinal column support system in accordance with the invention are the subject matter of the dependent Claims.

A spinal column support system in accordance with the invention thus comprises a bone screw which is formed so as to be able to move in an axial manner above the bone screw shaft, a plate or rod arrangement having at least one opening in which the bone screw is screwed, as well as an upper and a lower disk that are accommodated in the plate or rod at a spaced distance one above the other so as to be able to be displaced and positioned and that each comprise a hole, through which the bone screw passes.

The upper and/or lower disks are provided with eccentric, i.e., not disposed on the central axis of the disks, holes, for example a circular bore and are attached so as to be rotatable. For this purpose circular disks are, in an expedient manner, inserted and freely attached in the units on the upper and lower sides of the plate or rod.

The lower disk advantageously contains a conical bore that is not located in the central axis thereof.

Owing to the free moveability of the disks and to the rotation thereof, the bores of the upper and lower disks can be attached in a perpendicular manner one above the other such that the upper part of the bone screw can be inserted in a perpendicular manner.

Owing to the fact that the bone screw is formed so as to be able to move axially above the bone screw shaft, the bone screw can always be attached by rotation or inclination, in a perpendicular manner, in the plate or rod system when the bone screw shaft is located in an imprecisely defined position in the vertebra of the patient. In this manner, the bone screws can stabilize the vertebrae bones in a tension- and force-free manner with respect to each other.

A vertebrae support system in accordance with the invention is thus formed as a plate having suitable openings and transverse stabilizers, so that the bone screws to be attached can be fixedly screwed, regardless of how they protrude from the vertebrae bone, using two circular disks in which eccentric circular holes are located. The bone screws themselves are additionally formed so as to be multi-axial in the upper part and thus permit extreme inclined positions of the vertebrae bone screws.

The plate openings are designed in an expedient manner such that they consist of a rectangle or square, with the short sides in each case being replaced by semicircles (longitudinal hole), that are located on the lower side and on the upper side of the plate.

In one advantageous exemplified embodiment of the spinal column support system in accordance with the invention, the bone screws are formed in a multi-axial manner and an adjusting screw forming the upper part of the bone screw is coupled to the bone screw via a ball bearing system. The adjusting screw is always inserted into the plate system in a perpendicular manner, wherein the adjusting screw can be inserted by freely attaching the upper and lower circular disks in a force-free manner. By tightening the nuts, the entire system can be coupled and adapted in a force-free manner to the vertebrae to be supported and attached.

A second type of bone screw is practically identical with the exception that in the screwed condition the vertebrae bone still has a defined degree of residual movement.

In contrast to the current systems found on the market, wherein the bone screws always have to be screwed into the vertebrae bones in a precise linear alignment, so that a screw connection via a rod system or a narrow slot-like plate is generally possible, the plate in accordance with the invention and the screws associated therewith ensure that even with extreme exiting positions of the vertebrae bone screws, these screws can be fixedly screwed into the plate without even the smallest additional application of force.

A preferred embodiment of the invention is characterized in that the support plate is assembled from modularly identical functional units. This means that according to the number of vertebrae to be supported, the plate can be extended by identical units. This produces a very stable form with zones acting in a defined resilient manner. The plate elements are formed such that the shape of the openings with their inner-lying support ring receive the upper round disk and the lower round thicker disk and can be arbitrarily displaced and positioned therein. By rotating the lower disk (followed by the upper disk), the conical hole located therein can be freely positioned in order to insert the adjusting screw. When the nut is tightened, the spherical head of the bone screw as well as the upper plate and the lower plate are fixedly clamped to the inner-lying support ring. The lower and the upper disks can then no longer be displaced. The bone screw is likewise attached, wherein the spherical coupling element between the bone screw and adjusting screw is locked by inserting the adjusting screw into the conical bore of the lower disk.

Accordingly, the surgeon can attach the placed bone screws in the plate system without applying forces onto the vertebrae system.

A further preferred embodiment of the stabilizer in accordance with the invention is the so-called semi-rigid spinal column support system. This consists of the fact that the bone screws have a calotte system disposed directly behind the multi-axial joint, which calotte system permits the front part of the bone screw and thus the part screwed into the bone to have conical or pyramid-like free movement. This small amount of free movement for the supported vertebrae stimulates the circulation of blood, which has a positive effect on the healing process. The calotte joint is in an ideal manner disposed directly on the vertebrae joint and supports this latter in a moveable manner in a restricted region. The calotte element also preferably contains a stepped torsion protector.

### BRIEF DESCRIPTION OF THE DRAWINGS

FIG. 1 shows the plan view of a first exemplified embodiment of a spinal column support system in accordance with the invention in a rigid embodiment;

FIG. 2 shows a lateral view of the spinal column support system of FIG. 1;

FIG. 3 shows the plan view of a second exemplified embodiment of a spinal column support system in accordance with the invention in a semi-rigid embodiment;

FIG. 4 shows a lateral view of the spinal column support system of FIG. 3.

### DESCRIPTION OF PREFERRED EXEMPLIFIED EMBODIMENTS

The invention will be described in more detail hereinafter with the aid of two exemplified embodiments. However, this illustration does not serve to limit the invention to the definitively described combination of features, nor is the invention to be limited to the combination of features provided in the dependent Claims.

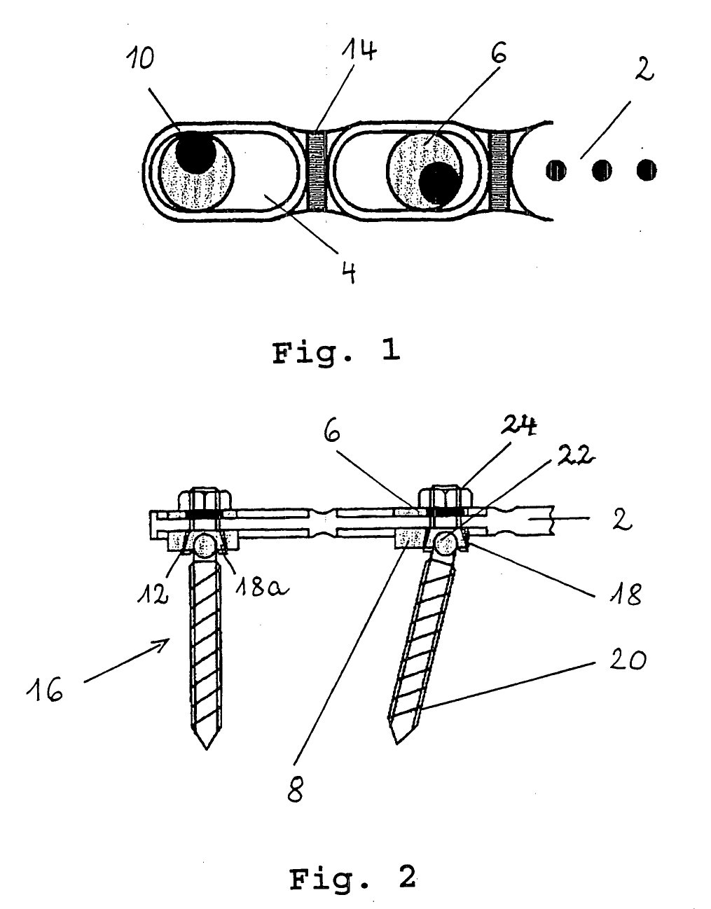

Reference is first made to FIGS. 1 and 2 that show a first exemplified embodiment in the form of a rigid spinal column support system. A plate 2 comprises an elongated opening 4. Two circular disks, an upper disk 6 and a lower, thicker disk 8, disposed one above the other at a spaced distance, are accommodated in the opening 4 in a longitudinally-displaceable and rotatable manner. The disks 6, 8 each comprise eccentrically disposed holes 10, 12, the lower one of which is formed in a conical manner, i.e., it is wider at the bottom. Resilient zones that are formed as a bending point 14 are located between such units of the plate 2.

A bone screw 16 having an upper part 18 and a lower part 20 that is formed in a moveable manner with respect to the upper part is guided through the holes 10, 12 and is attached, i.e., fixedly screwed, in the region of the upper part. The upper part of the bone screw 16 is formed as an adjusting screw 18 with an adjusting body 18a in the illustrated exemplified embodiment. The lower part of the bone screw 16 is the actual bone screw shaft 20. On the upper end, the bone screw shaft is provided with a spherical head 22 that is accommodated in the adjusting body 18a in a rotationally movable manner. A nut 24 serves to attach the arrangement, which nut on the one hand fixedly secures the spherical head 22 in the adjusting body 18a and on the other hand fixedly secures the two disks 6, 8 in the plate 4.

By displacing and rotating the two disks 6, 8 for aligning the spinal column support system after insertion of the bone screws into the vertebrae bones, it is possible to compensate for inclined positions of the bone screw, i.e., of the bone screw shaft 20. The fact that the bone screw 16 with its two parts 18, 20 is disposed so as to be multi-axial imparts additional flexibility to the arrangement.

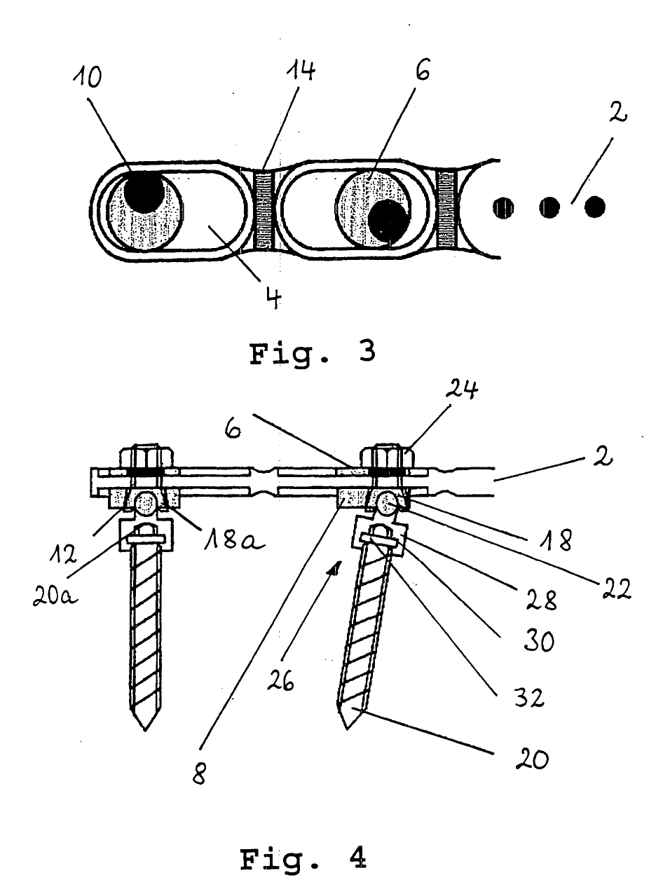

A second exemplified embodiment of the invention in the form of a semi-rigid spinal column support system will be described with the aid of FIGS. 3 and 4. The parts which are the same as those in the first exemplified embodiment will not be described again.

In this exemplified embodiment, the bone screw shaft 20 does not terminate directly in the spherical head 22 but rather there is additionally provided a holding device (holding element) 26 that is formed as a calotte system and in turn is disposed in a moveable manner. The holding device 26 terminates at its upper end in the spherical head 22 and consists of a calotte body 28 that comprises a calotte bearing 30 on the lower side. The upper end 20a of the bone screw shaft 20 is mounted in the calotte bearing 30 in a conical or pyramid-like freely moveable manner. A stepped torsion protector 32 is provided in the calotte body 28.

Owing to the additional freedom of movement of the supported vertebrae that is possible to a small extent, circulation of blood is stimulated and the healing process is accelerated.

1. Spinal column support system comprising a bone screw (16), a plate or rod arrangement having at least one opening (4) in which the bone screw (16) is displaceable, an upper and a lower rotatable fixing element that can be positioned at a spaced distance one above the other with respect to the plate (2) or rod and that each comprise an eccentrically disposed hole (10, 12), through which the bone screw (16) passes, characterized in that the bone screw (16) is formed so as to be able to move axially above the bone screw shaft and an upper and a lower disk (6, 8) are each provided as the fixing elements, which disks are accommodated in the plate (2) or rod in a displaceable manner and each comprise an eccentrically disposed hole (10, 12).

2. Spinal column support system according to claim 1, characterized in that the plate or rod is provided with a support ring in the inner wall of the opening, which support ring accommodates the upper and the lower disks.

3. Spinal column support system according to claim 1, characterized in that the upper and the lower disks (6, 8) are circular.

4. Spinal column support system according to claim 1, characterized in that the lower disk (8) is thicker than the upper disk (6).

5. Spinal column support system according to one of claims 1 through 4, characterized in that the hole (12) of the lower disk (8) is conical.

6. Spinal column support system according to claim 1, characterized in that the bone screw (16) consists of an upper part (18) that is provided for screwing in the plate (2) or rod and comprises a spherical receptacle on the lower end, and of a lower bone screw shaft part (20) that comprises, on the upper end, a spherical head (22) that is accommodated in the spherical receptacle in a rotationally moveable manner.

7. Spinal column support system according to claim 6, characterized in that the bone screw shaft part (20) is provided with a holding element (26) on the upper end, which holding element comprises, on the upper end, the spherical head (22) accommodated in the upper bone screw part in a rotationally moveable manner and also comprises a calotte bearing (30) beneath the spherical head for receiving the upper end (20a) of the bone screw shaft part (20), in such a manner that the bone screw shaft part is freely movable in the calotte bearing in a conical or pyramid-like manner.

8. Spinal column support system according to claim 7, characterized in that a stepped torsion protector (32) is provided in the calotte bearing (30).

9. Spinal column support system according to claim 1, characterized in that the upper part of the bone screw is an adjusting screw (18) that is provided with a thread on its end opposite the bone screw shaft (20) and can be attached by means of a nut (24).

10. Spinal column support system according to claim 1, characterized in that one or several units are provided as an extension for supporting one or several vertebrae.

11. Spinal column support system according to claim 10, characterized in that a bending zone (14) is provided between adjacent units.
